(19) United States
(12) Patent Application Publication
(10) Pub. No.: US 2012/0078251 A1
(43) Pub. Date: Mar. 29, 2012

(54) External Fixator Linkage

(75) Inventors: Vincent A. Benenati, Dix Hills, NY (US); Gregory S. Ahmad, Brightwaters, NY (US); Michael Vitale, Brooklyn, NY (US)

(73) Assignee: MGV Enterprises, Inc., Brightwaters, NY (US)

(21) Appl. No.: 12/888,443

(22) Filed: Sep. 23, 2010

(51) Int. Cl. A61B 17/00

(52) U.S. Cl. 606/56; 606/54

(57) ABSTRACT

Methods and apparatus are provided for coupling a spatial frame and a brace using a coupling device having a quick-release mechanism. The spatial frame may be secured to an appendage, and the brace may support the appendage. The coupling device may include a linkage that comprises: a mounting member adapted to be detachably coupled to the brace and to a fixator post adapted to be detachably coupled the spatial frame; a position fixing member adapted to adjustably immobilize the fixator post in a coupled position when the fixator post is coupled to the mounting member; and a quick-release mechanism adapted to detachably couple the mounting member to the brace; wherein the quick-release mechanism is further adapted to be engaged and disengaged repeatedly by hand and without separate tools. Numerous other aspects are provided.

### CROSS REFERENCE TO RELATED APPLICATIONS

This application is related to U.S. patent application Ser. No. 11/509,461 (“the '461 application”), titled “External Fixator Linkage,” and filed 24 Aug. 2006, which is incorporated by reference herein in its entirety for all purposes.

### BACKGROUND

This invention relates to medical devices used to treat bodily limbs that have been compromised, such as due to breakage or surgery. More specifically, the invention relates to device assemblies and systems that immobilize limb portions and brace adjacent joints. In particular, exemplary embodiments of the invention include an external fixator linkage that interconnects an external fixator and a brace.

Circular fixation allows precise and dependable correction of limb fracture and deformity (e.g., limb length discrepancy). For instance, external fixation of the tibia and femur allows rigid and precise control of lower extremity fractures and deformity correction. External fixation implies transfixation of muscles and tendons with screws that traverse the skin into the bone. External fixators commonly are in place for four to nine months.

However, circular fixation also carries a number of risks, most notably joint stiffness and muscular contracture, which may continue even with physical therapy. Although numerous means have been employed to maintain joint motion and keep muscles and tendons at length, physical therapy is the main preventive measure, but physical therapy nonetheless may not prevent such problematic contracture. Joint stiffness and muscular contracture often limit the amount of correction that is possible, particularly in cases involving leg lengthening. Correction of muscular contracture may be difficult, if not impossible, without further surgery (e.g., joint mobilization, tendon lengthening). In some cases, a fixator may be extended to a subsequent segment (e.g., the foot) to prevent further contracture.

Braces have been developed which allow for controlled motion of a joint adjacent to a limb that is circularly fixated to prevent joint stiffness and muscular contracture. Bracing can play an important role in preventing the development of these problems. These braces commonly use screwed-on linkages to circular fixators to enhance stability around the joint and restrict motion that may inhibit the healing process. However, experience has shown that it is extremely difficult to adequately fit the brace to the leg with circumferential rings, and extremely difficult to maintain tendon length without having an adequate place to affix to the body.

### SUMMARY

In a first aspect of the invention, a linkage is provided for coupling a spatial frame to a brace adapted to support an appendage. The linkage includes a mounting member, a position fixing member, and a quick-release mechanism. The mounting member is adapted to be detachably coupled to the brace and to a fixator post adapted to be detachably coupled the spatial frame. The position fixing member is adapted to adjustably immobilize the fixator post in a coupled position when the fixator post is coupled to the mounting member. The quick-release mechanism is adapted to detachably couple the mounting member to the brace. The quick-release mechanism is further adapted to be engaged and disengaged repeatedly by hand and without separate tools.

In a second aspect of the invention, a system is provided that includes a spatial frame, a brace, a fixator post, and a linkage for coupling the fixator post to the brace. The spatial frame is adapted to be secured to an appendage. The brace is adapted to support the appendage. The fixator post is adapted to be coupled to the spatial frame and to the brace. The linkage includes a mounting member, a position fixing member, and a quick-release mechanism. The mounting member is adapted to be detachably coupled to the brace and to the fixator post. The position fixing member is adapted to adjustably immobilize the fixator post in a coupled position when the fixator post is coupled to the mounting member. The quick-release mechanism is adapted to detachably couple the mounting member to the brace, and the quick-release mechanism is further adapted to be engaged and disengaged repeatedly by hand and without separate tools.

In a third aspect of the invention, a device is provided that includes a brace fixator and a linkage. The brace fixator is mountable to a brace. The linkage is adapted to be coupled to the brace fixator and to a fixator post. The fixator post is adapted to be coupled to a spatial frame that is distal to the brace. The linkage includes a block, a locking mechanism, a handle, and a quick-release mechanism. The locking mechanism is adapted to secure the fixator post to the block. The quick-release mechanism is adapted to detachably couple the linkage to the brace fixator. The quick-release mechanism is further adapted to be engaged and disengaged repeatedly by hand and without separate tools.

In a fourth aspect of the invention, a method is provided for detachably coupling a brace and a fixator post using a coupling device. The method includes the actions of using a position fixing member to immobilize a fixator post on the coupling device and using a quick-release mechanism to detachably couple the coupling device to the brace. Using the position fixing member may include slidably receiving the fixator post in a channel in the coupling device and engaging a locking mechanism to lock the fixator post in the channel. Using the quick-release mechanism may include engaging a pin to retract a catch on the quick-release mechanism, inserting the quick-release mechanism into the brace, and disengaging the pin to allow the catch to extend to engage the brace.

Other features and aspects of this invention will become more fully apparent from the following detailed description, the appended claims and the accompanying drawings.

### BRIEF DESCRIPTION OF THE DRAWINGS

Features of the invention can be more clearly understood from the following detailed description considered in conjunction with the following drawings, in which the same reference numerals denote the same elements throughout, and in which:

FIG. 1 shows a perspective view of an exemplary embodiment of a limb brace system according to aspects of the invention.

FIG. 2 shows a first perspective view of an exemplary embodiment of a frame-brace coupling device according to additional aspects of the invention.

FIG. 3 shows a second perspective view of the exemplary embodiment of the linkage for a frame-brace coupling device of FIG. 2.

FIGS. 4A and 4B show perspective views of an exemplary embodiment of a foot/ankle brace system according to still further aspects of the invention.

### DETAILED DESCRIPTION

The invention relates to an external fixator linkage. The exemplary embodiments of the invention describe frame-to-brace linkages, and more generally, coupling devices that may detachably couple a spatial frame to a limb brace, such as via circulator fixators. The invention involves a dynamic brace that may be affixed directly to either a unilateral external fixator or a multilateral external fixator, allowing for a more customized and/or more precise positioning of a joint center over a center of a knee or ankle joint. Exemplary embodiments include a multiaxial connector that allows six degrees of freedom and accommodates a wide range of patient sizes, bone deformities, and positioning of the external fixator on the leg. Rigid fixation to the frame allows for generation of requisite torque to keep the joint moving.

As will be described further below, detaching the coupling device may allow the brace to be removed for physical therapy; washing the patient; repairing, replacing, or cleaning the brace; etc. The coupling device also allows the limb brace to be properly aligned and fitted to the patient. As such, the coupling device may provide a therapeutic effect to alleviate and/or prevent muscular contracture, joint stiffness and any other negative effects associated with circular fixation. In exemplary embodiments of the invention, the coupling device comprises a quick-release mechanism that allows the brace to more easily be donned and removed by the patient, such as for washing and other hygiene-related activities. By combining a quick-release mechanism with a multiaxial connector, a patient may more easily apply, position, adjust, and remove the brace, as needed.

In this context, “quick-release” connotes a detachment process that may be accomplished repeatedly, by hand, without separate tools, and using moderate force. Likewise, “quick-release” implies a similar attachment process that may be accomplished repeatedly, by hand, without separate tools, and using moderate force. Operable without separate tools, a quick-release mechanism avoids the need for the patient to carry separate tools (e.g., a handheld screwdriver, plyers, or wrench) that otherwise might be needed to apply, position, adjust, and/or remove the brace. To operate the quick-release mechanism, the patient need only have one or two hands capable of moderate force, and the hand(s) may belong to the patient or an assistant.

With respect to repeatability, a quick-release mechanism may be engaged and disengaged repeatedly without material change or deformation of mechanism components that might render the mechanism a single-use mechanism. In contrast to a quick-release mechanism, as used herein, a single-use mechanism may be engaged and disengaged only once, and the processes of engagement and disengagement leave the single-use mechanism unable to be engaged a second time. Mechanical crimping, for example, would be a single-use mechanism.

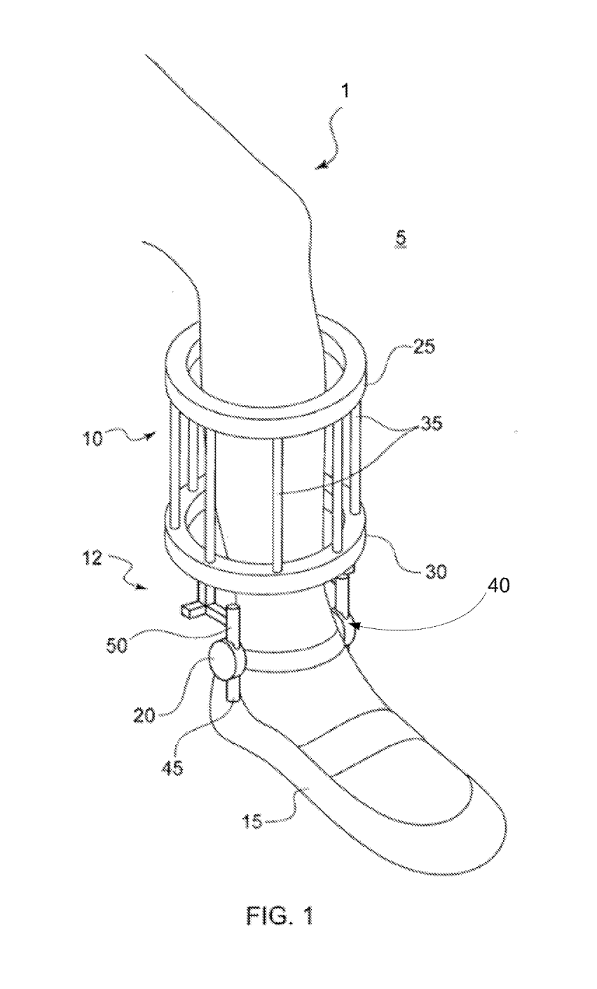

FIG. 1 shows a limb 1 fitted with an exemplary embodiment of a limb brace system 5 according to aspects of the invention. The limb brace system 5 generally may comprise an external fixator (e.g., a spatial frame 10) mounted on limb 1, a coupling device 12, a brace 15, and a joint 20, wherein coupling device 12 connects spatial frame 10 to brace 15 via joint 20. Spatial frame 10 may be a Taylor Spatial Frame or Ilizarov-Taylor Spatial Frame, which comprises a proximal circular fixator 25 and a distal circular fixator 30 coupled together via a plurality of struts 35. Although not shown, it is known in the art that spatial frame 10 may be secured to limb 1 using, for example, pins, screws, rods, wires, etc. to prevent rotational and/or translational movement of spatial frame 10 relative to limb 1.

Spatial frame 10 may be used to support a fractured limb and/or correct a deformity (e.g., limb length discrepancy) through the concept of callotasis. In the latter instance, a surgical fracture may be created in a long bone in limb 1. The bone is allowed to commence healing but is distracted using spatial frame 10 to regenerate new bone for correcting the deformity, e.g., lengthening the bone. Spatial frame 10 may also be used for a bone transplant. In this case, a defect in a long bone may be treated by removing a segment of bone while simultaneously lengthening the bone to replace the removed segment and produce a single bony unit. Regardless of the reason for use, spatial frame 10 may be mounted over any long bone, e.g., femur, tibia, humerus, ulna.

As shown in FIG. 1, brace 15 may be a boot worn on the foot when the spatial frame 10 is mounted on a lower leg. However, a structure of brace 15 may be determined based on the body part on which it will be worn. For example, when brace 15 is to be attached to a lower leg (e.g., spatial frame 10 is mounted on an upper leg), brace 15 may be a wrap, a circular band, a cuff, a shell, etc. When brace 15 is to be attached to the hand (e.g., spatial frame 10 is mounted on a lower arm), brace 15 may be a glove.

Joint 20 allows brace 15 to move relative to spatial frame 10. For example, joint 20 may comprise a pair of hinges 40 that have distal arms 45 coupled to the brace and proximal arms 50 coupled to coupling device 12 (coupled to spatial frame 10). Thus, in the exemplary embodiment shown in FIG. 1, joint 20 may function substantially similarly to an ankle joint, allowing extension and flexion of the foot about joint 20. This embodiment may be similarly implemented over a knee joint. For example, spatial frame 10 may be mounted on the upper leg (over the femur) and brace 15 may be worn on the lower leg. The joint 20 may then function substantially similarly to a knee joint, allowing extension and flexion of the lower leg about joint 20. The system 5 may also be implemented on limbs of the upper body. While joint 20 is shown as a hinge joint only allowing movement in one plane, those of skill in the art should recognize that a rotational or sliding joint may be utilized to simulate other body joints and/or other degrees of movement.

In the exemplary embodiment, hinges 40 of joint 20 may be configured for operation in a static mode or a dynamic mode. In the static mode, an angle between proximal arms 45 and distal arms 50 may be fixed to create a preselected angle between brace 15 and spatial frame 10. That is, after a surgical procedure, brace 15 may be set in a predetermined position creating an initial angle between spatial frame 10 and brace 15. Increasing the initial angle to the preselected angle may stretch muscles in the lower leg and foot, alleviating and/or preventing joint stiffness and muscular contracture.

In the dynamic mode, a continuous pressure may be applied in a predetermined direction to which the patient may apply resistance. For example, if hinges 40 are configured to apply pressure to cause extension of the foot, the patient may resist the pressure by attempting to flex the foot. The pressure applied by hinges 40 may be variable, allowing the patient to gradually rebuild and then maintain muscle tone while wearing spatial frame 10.

Those of skill in the art will understand that various mechanisms may be utilized to implement a dual mode joint as described above. For example, a gearing mechanism having a ratchet may be used to implement the static mode. As the angle between spatial frame 10 and brace 15 increases, the ratchet may interlock with a gear to maintain the angle (i.e., resist muscle contracture pulling brace 15 back to the initial angle). The dynamic mode may be implemented by spring-loading the gearing mechanism and/or adding resistance bands thereto.

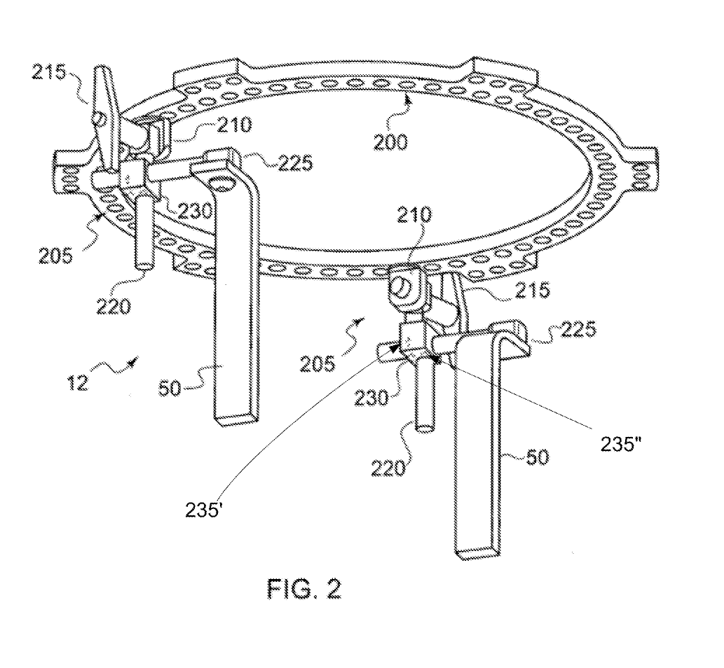

FIG. 2 shows a perspective view of an exemplary embodiment of coupling device 12 according to the invention. As noted above, coupling device 12 may be coupled to spatial frame 10 and joint 20. Coupling device 12 allows joint 20 and brace 15 to be removed from the patient, enabling brace 15 to be cleaned, the area previously covered by joint 20 and brace 15 to be washed, a physical therapist to easily remove joint 20 and brace 15, etc. Coupling device 12 also allows a distance between spatial frame 10 and joint 20 to be varied for properly aligning joint 20 with bodily joint (e.g., ankle, knee, etc.).

Coupling device 12 may include a circular fixator 200 which may be substantially similar to distal circular fixator 30 on spatial frame 10. In an exemplary embodiment, circular fixator 200 is coupled to distal circular fixator 30 by, for example, mechanical means (e.g., bolts, screws, pins, latches, etc.).

Extending from circular fixator 200 is a pair of linkages 205. Linkages 205 may be disposed on a circumference of circular fixator 200 so that they are separated by a distance that corresponds to a distance separating proximal arms 50 of joint 20. Linkages 205 may be used to detachably couple spatial frame 10 to joint 20. While the exemplary embodiment depicts linkages 205 as attached to circular fixator 200, those of skill in the art will understand that linkages 205 may be attached directly to distal circular fixator 30 of spatial frame 10.

In the depicted exemplary embodiment, linkages 205 include mounting members (e.g., L-brackets 210) that are coupled to circular fixator 200. Although the L-brackets 210 are shown in FIG. 2 as mechanically coupled to circular fixator 200 via a mechanical means (e.g., a bolt, a pin, etc.), those of skill in the art will understand that L-brackets 210 may be electrically or chemically affixed to circular fixator 200, and that this coupling may be temporary or permanent.

Holes are provided in L-brackets 210 for receiving quick-release mechanisms, which may include, for instance, pins 215. In the depicted exemplary embodiment, pins 215 are removably coupled to L-brackets 210. For example, each of the pins 215 may comprise a cylindrical portion having a catch which, when the catch is in a retracted position, the cylindrical portion may be passed through the hole. After the cylindrical portion has been passed through the hole, the catch may be released into an expanded position, preventing the cylindrical portion from retreating back through the hole. Control of the catch may be spring-loaded and affected using, for example, a button on a face of pin 215. For instance, depressing the button depresses a spring and allows the catch to move to the retracted position, whereas releasing the button releases the spring and moves the catch into the expanded position.

The pins 215 may be used to couple first elongate members 220 to L-brackets 210. When coupled to L-brackets 210, first elongate members 220 may be statically disposed and/or rotatable relative to L-brackets 210. For example, first elongate members 220 may be statically disposed after a surgical procedure to ensure that the surgical site heals properly, but a rotational aspect may be gradually introduced to prevent muscular contracture and joint stiffness. The rotational aspect may also be useful for properly aligning joint 20 with the bodily joint, as explained further below.

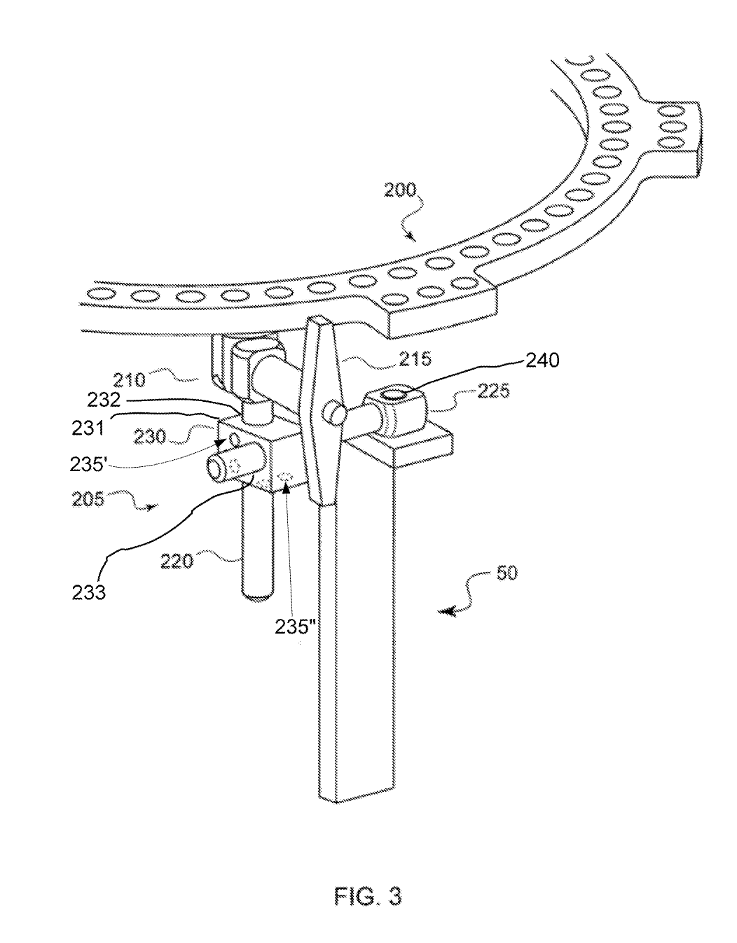

First elongate members 220 may be coupled to second elongate members 225 via position fixing members such as locking mechanisms 230. As shown more clearly in FIG. 3, locking mechanism 230 may be implemented as, for example, a sliding block 231 that includes a first channel 232 receiving first elongate member 220 and a second channel 233 receiving second elongate member 225. In the exemplary embodiment, first and second channels 232, 233 may be disposed at a predetermined angle (e.g., substantially perpendicular) relative to each other. A first lock 235′ may be disposed on first channel 232 to lock first elongate member 220 in a position relative to block 231, and a second lock 235″ may be disposed on second channel 233 to lock second elongate member 225 in a position relative to block 231.

After circular fixator 200 of coupling device 12 is affixed to distal circular fixator 30 of spatial frame 10, first and second elongate members 220, 225 may be moved relative to each other to align joint 20 with the corresponding bodily joint on limb 1. First and second elongate members 220, 225 may include stops to prevent disassociation with sliding block 231. When joint 20 has been properly aligned, first and second locks 235′, 235″ (e.g., locking screws) may statically position first and second elongate members 220, 225 relative to each other, maintaining joint 20 in its proper alignment (e.g., over the ankle, knee, elbow, etc.). Those of skill in the art will understand that various embodiments of locking mechanism 230 may be utilized to allow joint 20 to be properly aligned with the bodily joint.

Second elongate member 225 may be coupled to proximal arm 50 of joint 20 using, for instance, bolt 240. Coupling device 12 may be affected via mechanical means (e.g., a bolt, screw, etc.) so that coupling device 12 can be secured to joint 20 either temporarily or permanently.

In an exemplary use of system 5, spatial frame 10 may be mounted on the patient following a surgical procedure. For example, after lengthening one or more bones in the lower leg, spatial frame 10 may be mounted over the lower leg as is conventionally known in the art. Circular fixator 200 of coupling device 12 may then be affixed to distal circular fixator 30 of spatial frame 10. Brace 15 and joint 20 may then be mounted on the patient. With first and second elongate members 220, 225 traversing first and second channels 232 and 233 of block 231, second elongate members 225 may then be coupled to proximal arms 50 of joint 20, and joint 20 may be aligned with the ankle joint by positioning first and second elongate members 220, 225 relative to each other. When joint 20 has been properly aligned, first and second elongate members 220, 225 may be locked in their respective positions using locking mechanism 230.

With locking mechanism 230 securing first and second elongate members 220, 225, the quick-release mechanism of pins 215 facilitates quick attachment of first elongate member 220 to L-bracket 210 using bare hands and no separate tools. With pins 215 attaching first elongate member 220 to L-bracket 210, assembly of brace system 5 is complete, and the patient may resume limited use of limb 1. Disassembly of brace system 5 largely follows the reverse sequence, wherein pins 215 may be engaged to detach first elongate member 220 from L-bracket 210, and so on.

After system 5 has been fully mounted on the lower leg and foot, the patient or medical personnel may configure system 5 for therapeutic operation. As described above, joints 20 may be configured for the static mode or the dynamic mode to reduce the effects of joint stiffness and muscular contracture. In the static mode, the angle between spatial frame 10 and brace 15 may be set to a predetermined value, allowing the muscles, tendons and ligaments of the lower leg to be stretched. In the dynamic mode, joint 20 may be configured to apply pressure in a predetermined direction (plane, angle, etc.), forcing brace 15 to extend or flex. In this mode, the patient may resist the pressure strengthen/tone the muscles of the lower leg and foot.

In exemplary embodiments of the invention, the patient or medical personnel may remove brace 15 and joint 20. For example, when the patient is going to wash, during physical therapy, or when brace 15 and/or joint 20 needs to be cleaned, repaired, etc., pins 215 may be removed from L-brackets 210 on circular fixator 200. When pins 215 are removed, first elongate members 220 are released and joint 20 and brace 15 may be removed. Alternatively, first and/or second locks 235′, 235″ may be released, allowing sliding blocks 231 to be removed from first and/or second elongates members 220, 225, respectively. If sliding blocks 231 are removed from first and/or second elongate members 220, 225, preferably a marking device (e.g., pen, marker, scratch, etc.) is used to mark a position of first and/or second elongate members 220, 225 relative to sliding blocks 231.

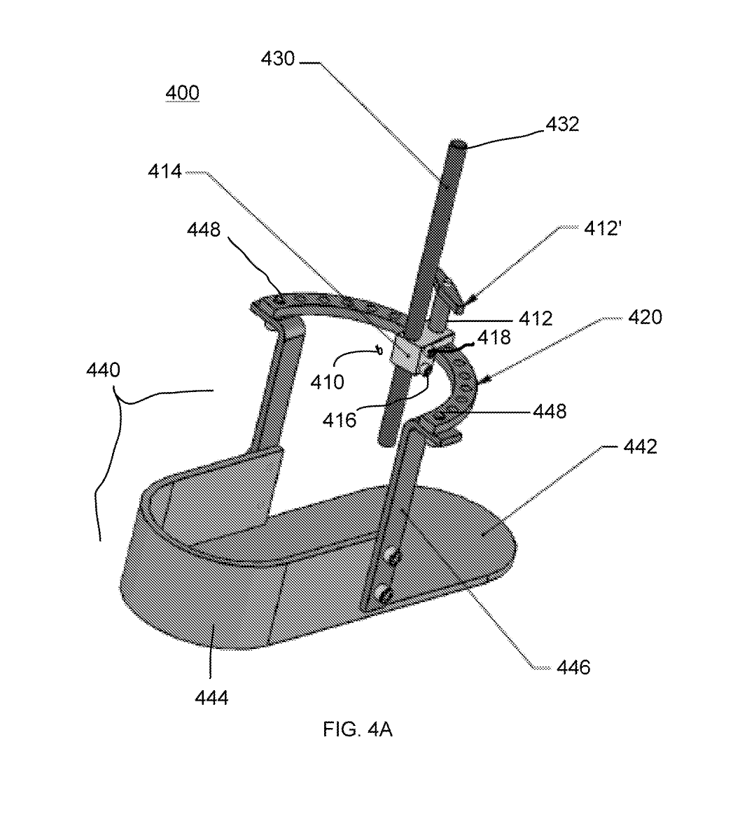

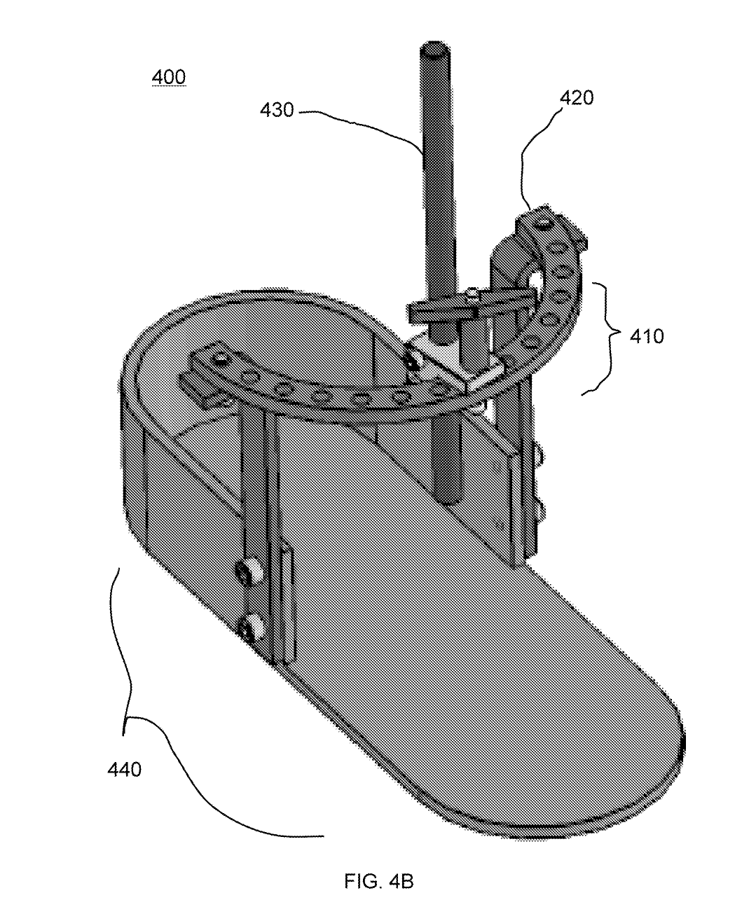

FIGS. 4A and 4B show perspective views of another exemplary embodiment of a limb brace system 400 according to the invention. As shown in FIGS. 4A and 4B, limb brace system 400 may be an assembly that includes a coupling device 410, a semicircular fixator 420 that may be coupled to coupling device 410, a fixator post 430 that also may be coupled to coupling device 410, and a brace 440 that may be coupled to semicircular fixator 420. In other embodiments, fixator 420 may be shapes other than semicircular; for instance, fixator 420 may be a full or partial oval or rectangle.

Semicircular fixator 420 may be called a brace ring in this embodiment, inasmuch as semicircular fixator 420 is a partial ring secured to brace 440, and in some embodiments, semicircular fixator 420 may be considered part of brace 440. In other embodiments, fixator 420 may be retrofitted to brace 440 to accommodate a connection with coupling device 410. In the embodiment of FIGS. 4A and 4B, for instance, fixator 420 may be considered a brace fixator, because fixator 420 is associated with brace 440, independent of a spatial frame. In contrast, proximal circular fixator 25 and distal circular fixator 30 each may be characterized as a spatial frame fixator because each is associated with a spatial frame, e.g., spatial frame 10, independent of a brace. Likewise, coupling device 410 also may be called a linkage or a brace coupling, in that coupling device 410 directly couples fixator post 430 to brace 440 via the brace ring, e.g., semicircular fixator 420.

Although not shown in FIGS. 4A and 4B, fixator post 430 may be coupled at a top end 432, distal to coupling device 410 and brace 440, to an external fixator, for instance, such as distal external fixator 30 of spatial frame 10 of FIG. 1, or circular fixator 200 of FIGS. 2 and 3. In some embodiments, fixator post 430 may be coupled to distal external fixator 30 using coupling device 12, or to circular fixator 200 using linkage 205, wherein fixator post 430 may replace first elongate member 220, or possibly second elongate member 225, depending on the configuration of limb brace system 400. In embodiments having fixator post 430 coupled to circular fixator 200 using linkage 205, pins 215 may provide a quick-release mechanism of securing linkage 205 to circular fixator 200, as in FIGS. 2 and 3. In other embodiments, fixator post 430 may be coupled to circular fixator 200 using a position fixing member similar to locking mechanism 230 comprised of sliding block 231.

As depicted in FIGS. 4A and 4B, brace 440 may comprise a foot brace that includes a foot plate 442, a heel cover 444 secured to foot plate 442, and brace uprights 446 secured to heel cover 444. In comparing the embodiment of FIGS. 4A and 4B to that of FIGS. 2 and 3, brace uprights 446 may be similar to proximal arms 50 inasmuch as both interconnect a brace and a fixator. However, brace uprights 446 may be different from proximal arms 50 in that FIGS. 2 and 3 show using a quick-release mechanism of pins 215 of linkage 205 connected to proximal arms 50 to detachably couple brace 15 to circular fixator 200, whereas FIGS. 4A and 4B show using bolts 448, e.g., a non-quick-release mechanism, to mount semicircular fixator 420 on brace uprights 446. Nonetheless, although not shown, pins 215 may be used instead of bolts 448 to secure semicircular fixator 420 to brace uprights 446 using a quick-release mechanism.

The embodiment of FIGS. 4A and 4B is shown as using a quick-release mechanism (e.g., pin 412) to secure coupling device 410 to semicircular fixator 420. Coupling device 410 includes pin 412, which may be similar to pins 215. Coupling device 410 also is shown to include a sliding block 414, and sliding block 414 may act as a mounting member for coupling to a fixator (e.g., semicircular fixator 420 in FIGS. 4A and 4B). In some embodiments, pin 412 may be secured to sliding block 414 and may serve as a handle 412′ of coupling device 410. Sliding block 414 may be traversed by fixator post 430, and, analogous to sliding block 231, sliding block 414 may comprise a locking mechanism 416 that include a lock 416′ disposed on a channel 418 to lock fixator post 430 in a position relative to block 414. In the embodiment of FIGS. 4A and 4B, locking mechanism 416 of sliding block 414 serves as a position fixing member.

In embodiments having fixator post 430 secured to a spatial frame or external fixator, as discussed above, the assembly of FIGS. 4A and 4B may be particularly advantageous in that only one quick-release linkage, e.g., coupling device 410, need be engaged or disengaged to attach or detach, respectively, a brace to or from the spatial frame or external fixator. Such an arrangement greatly facilitates the attachment/detachment processes, which hopefully would reduce a physical hurdle, as well as psychological hurdle, to cleaning and caring for the affected limb and brace.

The foregoing description discloses exemplary embodiments of the invention, which has been described with the reference to the exemplary embodiments. The specification and drawings, accordingly, should be regarded in an illustrative rather than restrictive sense. Various modifications and changes may be made to the embodiments without departing from the broadest spirit and scope of the invention. Modifications of the above disclosed apparatus and methods that fall within the scope of the invention will be readily apparent to those of ordinary skill in the art. Accordingly, additional embodiments may fall within the spirit and scope of the invention, as defined by the following claims.

1. A linkage for coupling a spatial frame to a brace adapted to support an appendage, the linkage comprising:
a mounting member adapted to be detachably coupled to the brace and to a fixator post adapted to be detachably coupled the spatial frame;
a position fixing member adapted to adjustably immobilize the fixator post in a coupled position when the fixator post is coupled to the mounting member; and
a quick-release mechanism adapted to detachably couple the mounting member to the brace;
wherein the quick-release mechanism is further adapted to be engaged and disengaged repeatedly by hand and without separate tools.

2. The linkage according to claim 1, wherein the spatial frame includes a spatial frame fixator, and the fixator post is adapted to be detachably coupled to the spatial frame fixator.

3. The linkage according to claim 1, wherein the brace comprises a brace fixator, and the mounting member is adapted to be detachably coupled to the brace fixator.

4. The linkage according to claim 1, wherein the quick-release mechanism comprises a pin, a handle, and a spring-loaded catch adapted to extend from and retract to the pin.

5. The linkage according to claim 1, wherein the position fixing member comprises a locking mechanism, and the locking mechanism includes a channel, adapted to slidably receive the fixator post in the channel, and a lock, adapted to immobilize the fixator post in the channel.

6. The linkage according to claim 5, wherein the position fixing member comprises a block, the channel comprises a bore formed through the block, and the lock comprises a screw threaded through the block and into the channel.

7. A system, comprising:
a spatial frame adapted to be secured to an appendage;
a brace adapted to support the appendage;
a fixator post adapted to be coupled to the spatial frame and to the brace; and
a linkage for coupling the fixator post to the brace, the linkage comprising:
a mounting member adapted to be detachably coupled to the brace and to the fixator post;
a position fixing member adapted to adjustably immobilize the fixator post in a coupled position when the fixator post is coupled to the mounting member; and
a quick-release mechanism adapted to detachably couple the mounting member to the brace;
wherein the quick-release mechanism is further adapted to be engaged and disengaged repeatedly by hand and without separate tools.

8. The system according to claim 7, wherein the brace comprises a brace fixator, and the mounting member is adapted to be detachably coupled to the brace fixator using the quick-release mechanism.

9. The system according to claim 8, wherein the brace fixator comprises a semicircular fixator.

10. The system according to claim 9, wherein the brace fixator is secured to brace uprights.

11. The system according to claim 10, wherein the brace comprises a foot brace, the brace uprights are secured to a heel cover, and the heel cover is secured to a foot plate.

12. The system according to claim 7, wherein the brace is one of a boot, a glove, a wrap, a shell, a band and a sleeve.

13. The system according to claim 7, wherein the spatial frame includes a circular fixator and a spatial frame linkage, the spatial frame linkage includes a second quick-release mechanism, and the spatial frame linkage is adapted to detachably couple the fixator post to the circular fixator.

14. The system according to claim 7, wherein the quick-release mechanism comprises a pin, a handle, and a spring-loaded catch adapted to extend from and retract to the pin.

15. The system according to claim 7, wherein the position fixing member comprises a locking mechanism, and the locking mechanism includes a channel, adapted to slidably receive the fixator post in the channel, and a lock, adapted to immobilize the fixator post in the channel.

16. The system according to claim 15, wherein the position fixing member comprises a block, the channel comprises a bore formed through the block, and the lock comprises a screw threaded through the block and into the channel.

17. A device, comprising:
a brace fixator mountable to a brace; and
a linkage adapted to be coupled to the brace fixator and to a fixator post;
wherein the fixator post is adapted to be coupled to a spatial frame that is distal to the brace;
wherein the linkage comprises a block, a locking mechanism, a handle, and a quick-release mechanism;
wherein the locking mechanism is adapted to secure the fixator post to the block;
wherein the quick-release mechanism is adapted to detachably couple the linkage to the brace fixator; and
wherein the quick-release mechanism is further adapted to be engaged and disengaged repeatedly by hand and without separate tools.

18. The device according to claim 17, wherein the quick-release mechanism comprises a spring-loaded catch adapted to extend from and retract to a pin, and the pin extends from the handle.

19. The device according to claim 17, wherein the block includes a channel for slidably receiving the fixator post, and the locking mechanism comprises a lock adapted to immobilize the fixator post in the channel.

20. The device according to claim 17, wherein the brace fixator comprises a semicircular fixator secured to the brace using brace uprights.
